(19) United States
(12) Patent Application Publication
(10) Pub. No.: US 2005/0101919 A1
(43) Pub. Date: May 12, 2005

(54) Device for an injector

(76) Inventor: Lennart Brunnberg, Tyreso (SE)

Correspondence Address: YOUNG & THOMPSON, 745 SOUTH 23RD STREET, 2ND FLOOR, ARLINGTON, VA, 22202, US

(21) Appl. No.: 10/702,692

(22) Filed: Nov. 7, 2003

(51) Int. Cl. A61M 5/315

(52) U.S. Cl. 604/197; 604/229

(57) ABSTRACT

The present invention relates to an injection device comprising a generally elongated tubular housing, a syringe containing medicament and having a needle, a needle shield slidably arranged to the housing and protruding a distance outside the front end of the housing, a plunger arranged to act on the syringe and a pre-tensioned drive member arranged the drive the plunger, characterized in that it comprises radially acting holding elements capable of releasably holding the plunger, axially acting actuator elements connected to the needle shield and capable of releasably locking the holding elements and axially acting activator elements capable of releasing the holding elements from the actuator elements have when the needle shield and the actuator elements has moved axially a certain distance due to that injection device has been placed against an injection site.

### TECHNICAL FIELD

The present invention relates to an injector for administering drugs in a safe and reliable way.

### BACKGROUND OF THE INVENTION

The handling and safety aspects of injector devices, having a certain degree of automatic functions, as well as immediate accessibility in emergency situations are issues that attract a lot of attention when developing this type of device.

A few of these devices have safety aspects like a two-step operation before the injection is activated in order to avoid unintended activation, for example if the user comes in contact with the trigger button before the injector is placed at the injection site. However many of these solutions are rather bulky and rely also on many components acting in co-operation and in sequence, one trigging another, which may lead to a mal-function, or that the device becomes complicated, hence not user friendly.

One device utilising a high degree of automation is described in the document WO 02/74774. The device is an auto-injector whereby the injection can be activated by a push button, i.e. penetration and injection, but only when the front end of the injector is pressed against the injection site. It is designed as a kind of two-step operation where the order has to be: pressing the injector at the site and then depressing the button. Any other order of sequence will not result in an activation of the device. This ensures that the injector cannot be accidentally activated by merely pushing the button nor even pushing the button and then pressing the front end.

Even though the device according to WO 02/74774 has proved to function well and displays a high degree of safety and user-friendliness there is always a desire for improvements of such devices, among them being the design of the mechanism in order to simplify the manufacture and assembly in order to reduce costs but at the same time maintain or even improve the reliability of the function of the device.

### SHORT DESCRIPTION OF THE INVENTION

The aim of the invention is to provide an injection device which is uncomplicated and easy to use, which is safe both before, during and after use and which displays a high degree of functionality.

According to one aspect of the invention, an injection device is provided comprising a generally elongated tubular housing, a container containing medicament and having a needle attached to the container, a needle shield slidably arranged to the housing and protruding a distance outside the front end of said housing, a plunger arranged to act on said container and a pre-tensioned drive means arranged to drive said plunger, characterised in that it comprises radially acting holding means capable of releasably holding said plunger, axially acting actuator means connected to said needle shield and capable of releasably locking said holding means and axially acting activator means capable of releasing said holding means from said actuator means when said needle shield and said actuator means has moved axially a certain distance because the injection device has been placed and pressed against an injection site.

According to another aspect of the invention it is characterised in that said holding means comprises generally radially extending inwards directed ledges and that said plunger comprises a groove, with mutual shapes as for the ledges to fit into said groove and that said ledges are arranged at the ends of flexible tongues.

According to a further aspect of the invention it is characterised in that it comprises a locking means capable of locking said activator means until said actuator means has moved axially a certain distance.

Preferably said locking means comprises hooks arranged on said activator means co-operating with fixedly arranged stop means and that said actuator means comprises means for displacing said hooks in relation to said stop means upon movement of said actuator means.

The injector according to the present invention comprises a number of radially and axially co-acting means in order to obtain a compact and yet reliable injecting device with rather few components. This implies that the holding means are acting to hold and lock the pre-tensioned plunger radially which is a safe way to lock and store the device pre-tensioned for long periods until use. As a benefit the user does not have to arm the device before use. The holding means are held in place by the axially slidable actuator means, preferably a sleeve, which in turn is connected to the needle shield. Thus the movement of the needle shield, when pressing the device against an injection site axially moves the actuator sleeve axially in relation to the holding means, but not so much that the holding means is released from the plunger. This step of releasing the plunger is performed by the activator means, for example a push button at the end of the device, which moves the holding means axially in relation to the actuator sleeve until the holding means is released and the plunger is released to act on the container.

The axial movement in order to release the holding means is thus performed both by the actuator sleeve but also by the holding means. There is thus a two-step operation in order to activate an injection. For enhancing the safety of the device it is designed so that the operation of the device is performed in a pre-determined sequence.

The needle shield is further provided with a spring that urges needle shield to an extended position surrounding the needle when the device is withdrawn from the injection site after the injection, and locking means for locking the needle shield in that extended position in order to avoid accidental needle sticks after injection and subsequently after disposal. The device may be provided with means for delivering a subsequent dose, whereby the needle first has to be removed and replaced. In order to enable this the needle shield can be released by a separate release mechanism in order to push it backwards, or with a separate tool, thereby exposing the needle so that it can be replaced.

The device may also be arranged for replaceable containers, i.e. when one container is emptied, the patient replaces it with a new container.

The mechanism of the injector of the present invention is designed such that an injection is only activated when first the injector is pressed against the injection site and then the activator (e.g. a button) is pressed or activated. It is not possible to press the activator first and then press the injector against an object to activate an injection nor is it possible to perform these steps simultaneously. Also if the injector is removed from an injection site before the activator is pressed, the injector is reset to its original locked state. This ensures a very high degree of safety in handling the device and prevention of faulty or not performed injections.

These and other aspects of and advantages with the present invention will become apparent from the following detailed description and from the accompanying drawings.

### SHORT DESCRIPTION OF THE DRAWINGS

In the following detailed description of the invention, reference will be made to the accompanying drawings, of which

FIG. 1A is a cross-sectional side view of one embodiment of the auto-injector according to the invention,

FIG. 1B is an enlarged view of FIG. 1A showing a locking part-of an actuator means of the device,

FIG. 1C is an enlarged view of a part of an activator means of the device,

FIG. 2A is a cross-sectional view of the front part of the auto-injector according to FIG. 1,

FIG. 2B is a cross-sectional view of the front part where the section is taken 90° in relation to the section of FIG. 2A,

FIG. 3 is an exploded view of the front part shown in FIGS. 2A and B,

FIG. 4A is a cross-sectional view of the rear part of the auto-injector according to FIG. 1,

FIG. 4B is a cross-sectional view of the front part where the section is taken 90° in relation to the section of FIG. 4A,

FIG. 5 is an exploded view of the rear part shown in FIG. 4,

FIG. 6A, B are side views of an actuating means comprised in the device according to FIG. 1,

FIG. 7 is a cross-sectional view of a holding means comprised in the device according to FIG. 1,

FIG. 8A, B cross-sectional views of the device when it has been pressed against an injection site, where the section of FIG. 8B is taken 90° in relation to FIG. 8A,

FIG. 8C is a detailed view of the activation means of the device,

FIG. 9A, B are cross-sectional views of the device when penetration has started, where the section of FIG. 9B is taken 90° in relation to FIG. 9A,

FIG. 9C is a detailed view of the position of the actuator means during this phase,

FIG. 9D is a detailed view of the activator means during this phase,

FIG. 10A, B are cross-sectional views of the device when injection has started, where the section of FIG. 10B is taken 90° in relation to FIG. 10A,

FIG. 11A, B are cross-sectional views of the device when injection is completed, where the section of FIG. 11B is taken 90° in relation to FIG. 11A,

FIG. 12A, B are cross-sectional views of the device when injector is removed from the injection site, where the section of FIG. 12B is taken 90° in relation to FIG. 12A,

FIG. 12C is a detailed view of the actuator means, and

FIG. 12D is a detailed view of activator means.

### DETAILED DESCRIPTION OF THE INVENTION

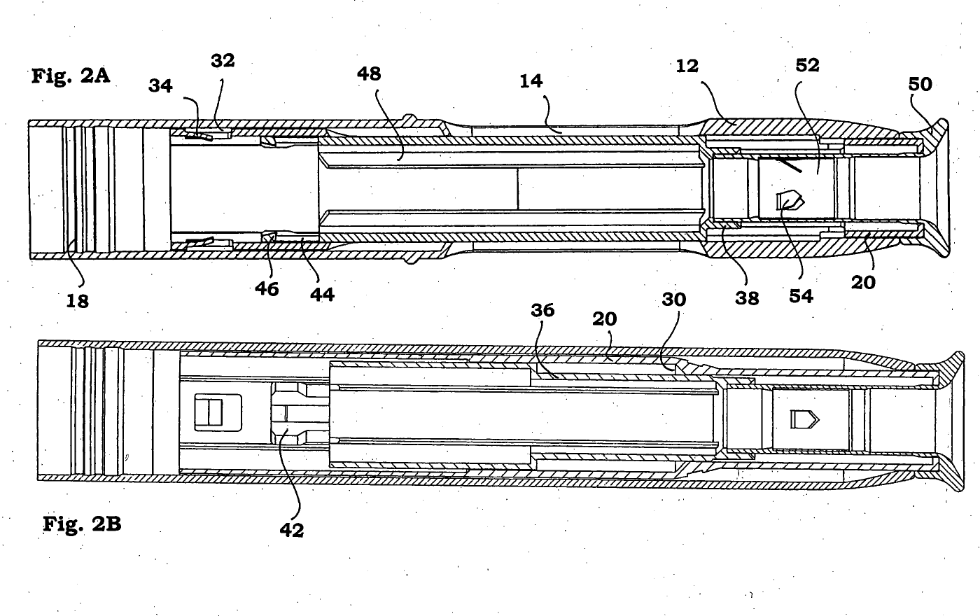

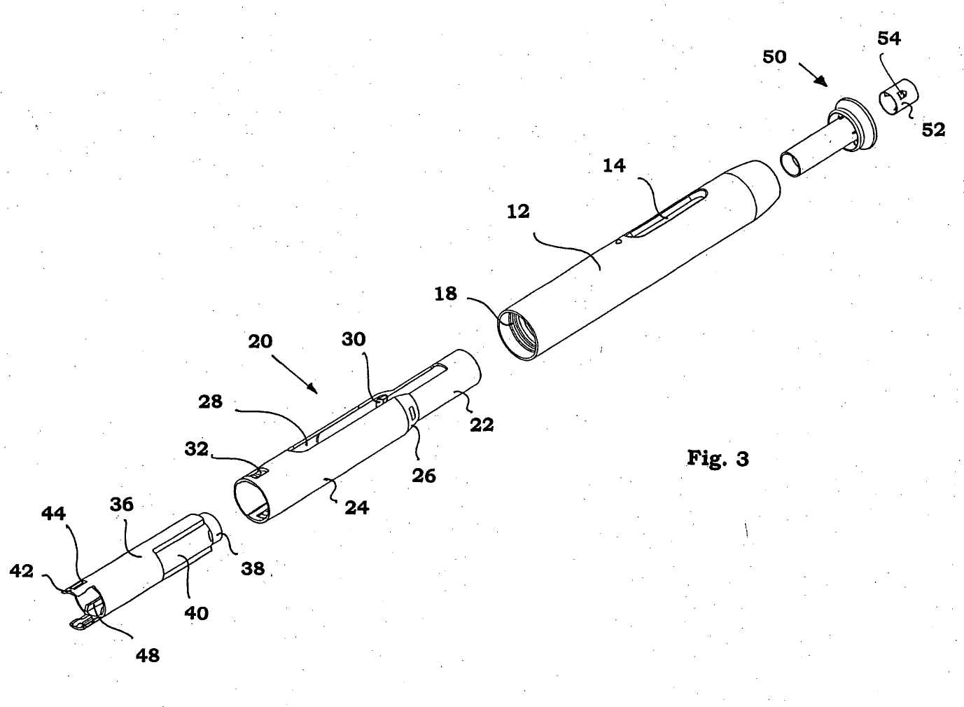

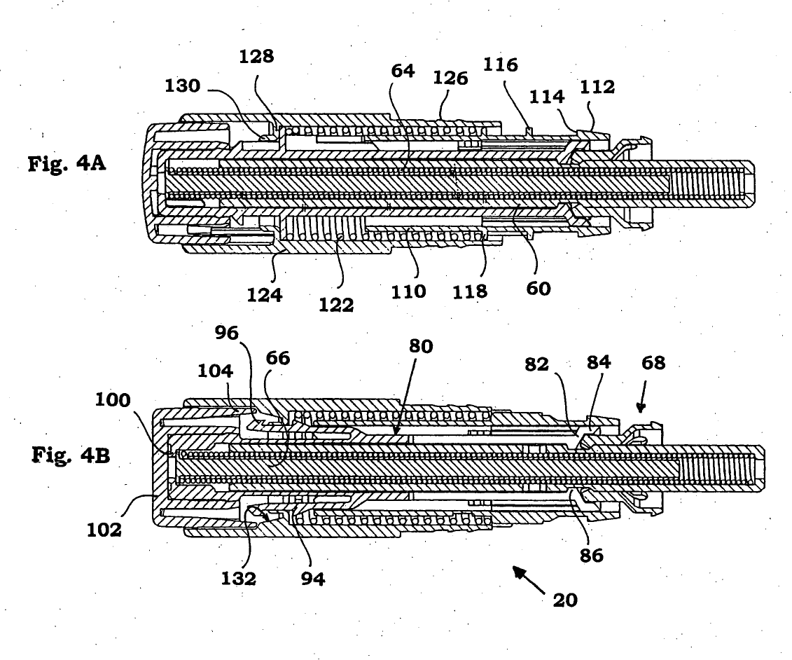

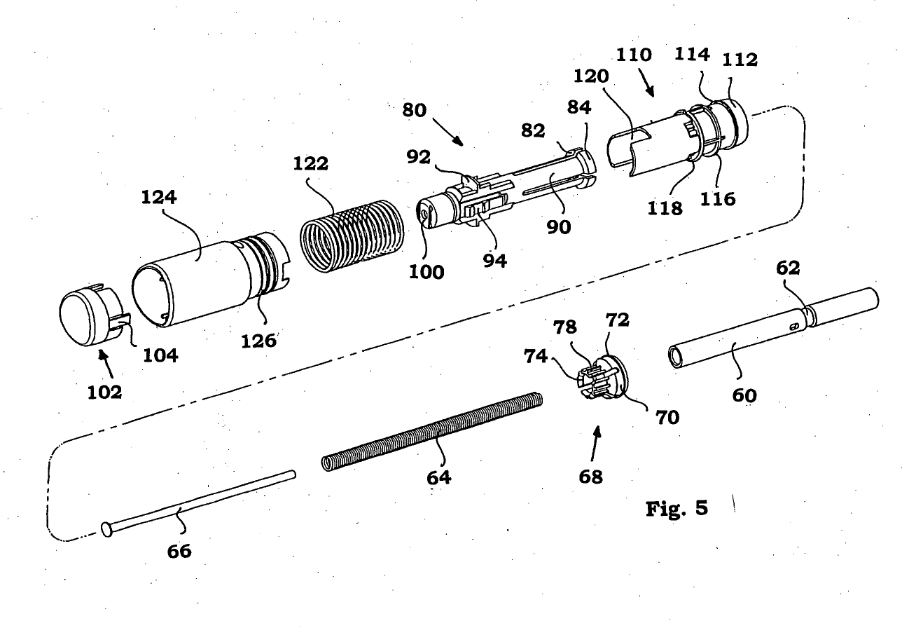

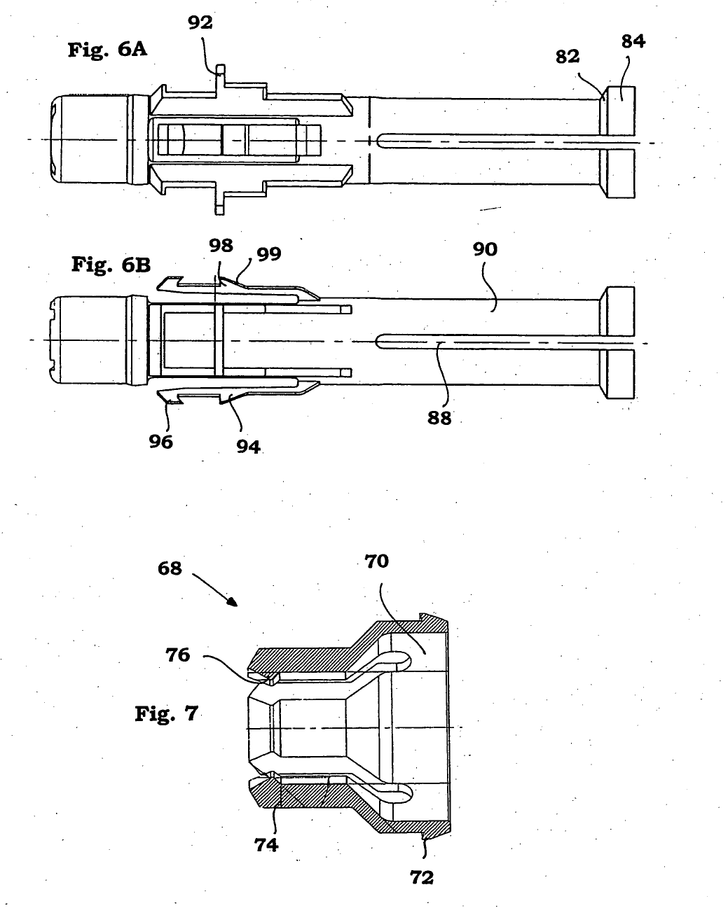

The embodiment shown in the drawings comprises a front part 10, FIGS. 2-3 and a rear part 60, FIGS. 4-5.

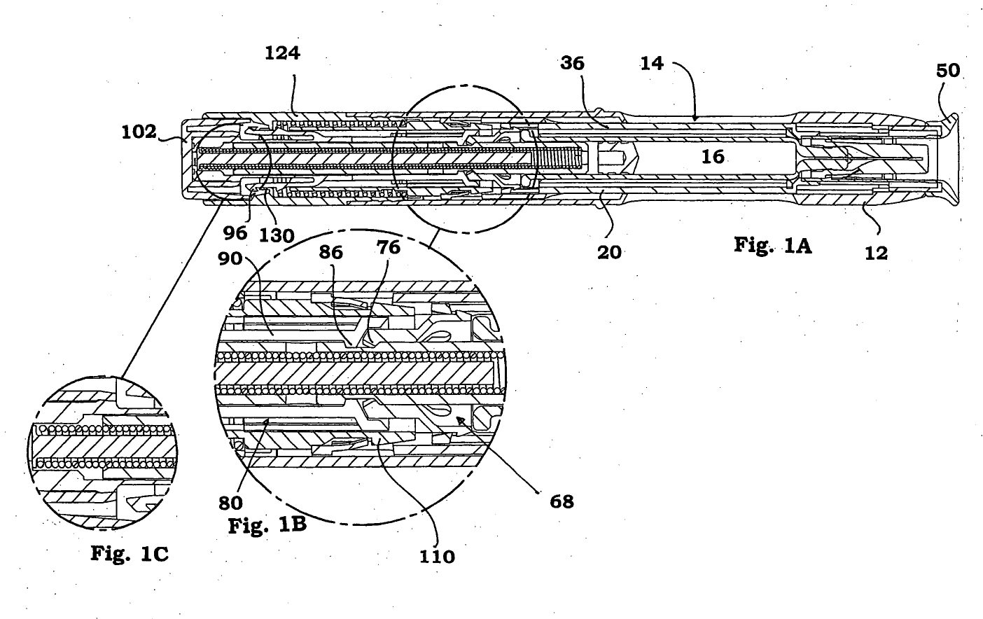

The front part 10 comprises a generally tubular front body 12 having elongated openings 14 for viewing a syringe 16, FIG. 1, and a somewhat narrowing front end. The rear end is arranged with annular recesses 18 on the inner surface. Inside the front body a needle shield 20 is slidably arranged. The needle shield is generally tubular with a first front part 22 having a certain diameter and a second rear part 24 having a diameter larger than the front part, where these parts are joined by an intermediate conical part 26, FIG. 3. Two elongated grooves 28 are arranged along the needle shield, on opposite sides of the needle shield, also for viewing the syringe. On the inner surface of the conical part a circumferential ledge 30 is arranged. At the rear end of the needle shield two openings 32 are arranged opposite each other, where each opening is arranged with somewhat inwardly projecting, flexible, tongues 34.

Further a syringe carrier 36 is arranged inside the needle shield in the form of a generally tubular body. The front part of the syringe carrier is arranged with a neck portion 38 of lesser diameter. Adjacent the neck portion cut-outs 40 have been made on either side to form guide surfaces. These surfaces cooperate with corresponding shapes of the inner surface of the needle shield in order to obtain a stop means against rotation of the syringe carrier relative the needle shield. The rear end of the syringe carrier is arranged with two rearwardly directed tongues 42 where each tongue is arranged with an opening 44 and an inwardly directed ledge 46 on the rear edge of each opening. The syringe carrier is further arranged with radially directed flanges 48 on its inner surface in order to obtain a space between the syringe carrier wall and a syringe to be placed inside.

At the front end of the front part a needle protection cap grabber 50 is arranged. It is inserted into the front part of the needle shield and held there by friction. Inside the cap grabber a metal ring 52 is arranged with sharp pointed tongues 54 directed somewhat inwards and towards the front end.

FIG. 4-5 shows the rear part or power unit 20 of the injector according to FIG. 1. It comprises a plunger 60 formed as a tube and with an outer diameter somewhat smaller than the inner diameter of the syringe body to be used. The plunger 60 is arranged with a circumferential groove 62 with a certain width. Inside the plunger a helical compression spring 64 is arranged and inside the spring a spring guide 66 is placed. Adjacent the groove of the plunger a holding member 68 is arranged. It comprises a ring-shaped body 70 FIG. 7, having an annular ledge 72 arranged around its circumference and a number of flexible tongues 74 directed towards the rear of the power unit, to the left in the figures. Each tongue 74 is arranged with inwardly directed ledges 76 arranged and shaped as to fit into the groove 62 of the plunger. Each tongue is further arranged with reinforcing ribs 78 on the outer surfaces.

Surrounding the plunger is an activator 80 with a mainly tubular shape. Its front end, to the right in the figures, has an inclined transition surface 82 which meets with a band-shaped part 84 with enlarged diameter. On the inner surface adjacent the transition surface an annular inwardly directed ledge 86 is arranged, with a shape as to fit into the groove 62 of the plunger. A number of longitudinally directed cut-outs 88 are arranged at the front part of the actuator so as to form flexible tongues 90. The activator is further provided with two stop ledges 92 directed radially outwards from the outer surface on either side. Between the stop ledges two flexible tongues 94 are arranged on the outer surface. Each tongue is arranged with an outwardly directed hook 96 at the outer end and a protrusion 98, with an inclined surface 99, a distance along each tongue. The upper end of the activator is arranged with an end wall 100. An activator button 102 is attached to the upper end of the activator having two tongues 104 attached to its outer edge and directed towards the front of the device.

Outside the activator an actuator sleeve 110 is slidably arranged, also of a generally tubular form. It comprises a front end with a conical part 112 ending in a ledge 114 on its outer surface. At a distance from the ledge a first annular ring 116 is arranged the outer surface. A second annular ring 118 is also arranged a further distance from the ledge. The rear end of the actuator sleeve is arranged with two oppositely arranged cut-outs 120 of a generally rectangular shape where the widths correspond to the width of the stop ledges 92 of the actuator. A compression spring 122, hereafter named needle shield spring is surrounding the actuator sleeve.

The previously named components of the power unit are housed in a rear housing 124 of a generally tubular shape, where the front end of the rear housing has a somewhat lesser diameter, corresponding to the inner diameter of the rear end of the front body and provided with a number of annular protrusions 126 which are intended to fit into the corresponding annular recesses 18 on the inner surface of the front body 12. Inside the rear housing an annular ring 128 is arranged, which ring is provided with a circumferential ledge 130 with a shape corresponding to the hooks of the actuator. Adjacent the annular ring 128 and in the vicinity of the tongues 104 of the activator button when placed in the housing 124 are arranged inclined surfaces 132, the function of which will be described below.

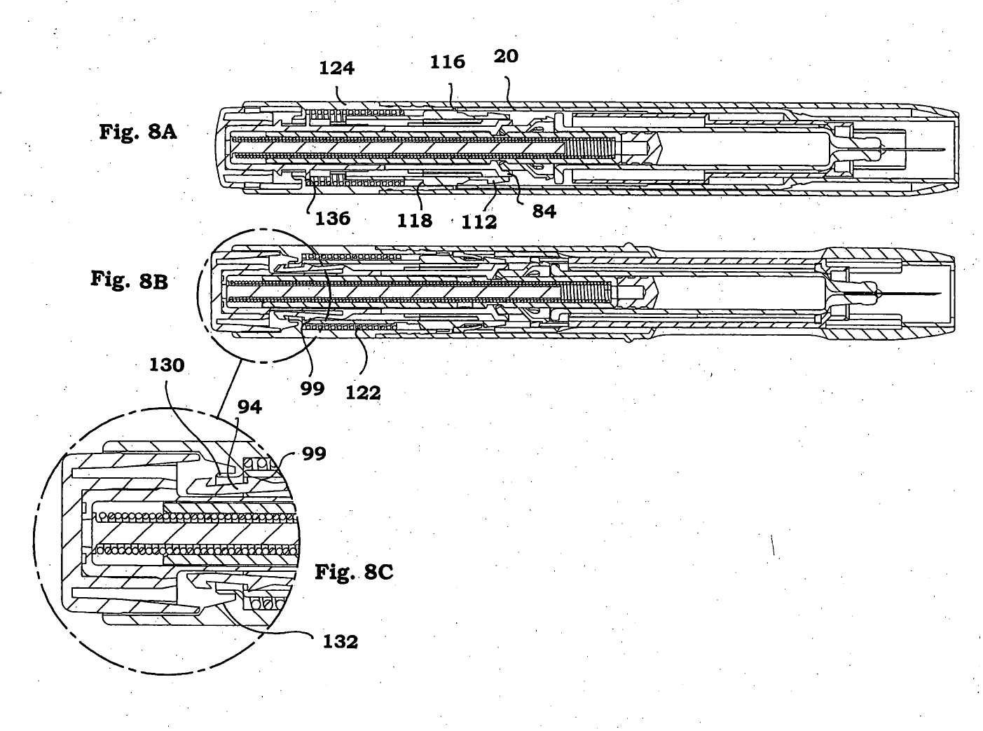

The function of the injector according to the invention will now be described in connection with the FIGS. 8-11.

When assembling the injector the front and the rear parts are assembled individually. As regards the power unit the plunger is held against the force of the compression spring in that the inwardly directed ledges 86 of the tongues 90 of the activator are situated in the groove 62 of the plunger 60 and that the actuator sleeve 110 prevents the tongues 90 from moving outwards. Further the ledges 76 of the holding member are also arranged in the groove 62, FIG. 1B. The hooks 96 of the activator are adjacent the circumferential ledge 130 as a second safety means should the tongues 90 move out of the groove of the plunger. In this position, if the activator button is depressed, it can only move a very short distance inwards together with the actuator 80 until the hooks engage the circumferential ledge.

A syringe is placed in the front end and a rear part is attached to the front part wherein the protrusions 126 fit into the recesses 18. At the same time the tongues 34 of the needle shield 20 fit behind the ledge 114 of the actuator sleeve 110 and the ledges 42 of the syringe carrier 36 pass behind the annular ledge 72 of the holding member 68. The needle protection cap grabber 50 is inserted into the front end of the device. The device is now ready for use.

When an injection is to be performed the needle protection cap grabber is pulled out of the injector. This causes the sharp pointed tongues 54 to be pushed into the rubber needle protection cap and remove it from the needle. The front end of the injector is then pressed against the injection site and the somewhat projecting front end of the needle shield is pushed into the housing, FIG. 8, against the force of the compression spring 122 acting between the second annular ring 118 of the actuator sleeve and a ledge 136 arranged inside the housing 124. The upper end of the needle shield is in contact with the first annular ring 116 of the actuator sleeve 110 and its movement causes the sleeve to move backwards, to the left in the figures, whereby a part of the band-shaped part 84 is situated outside the front part of the sleeve. The upper edge of the actuator sleeve will then come in contact with the inclined surface 99 of the tongues 94 on the actuator 80 whereby the hooks 96 are moved inwards and are free to pass inside the circumferential ledge 130.

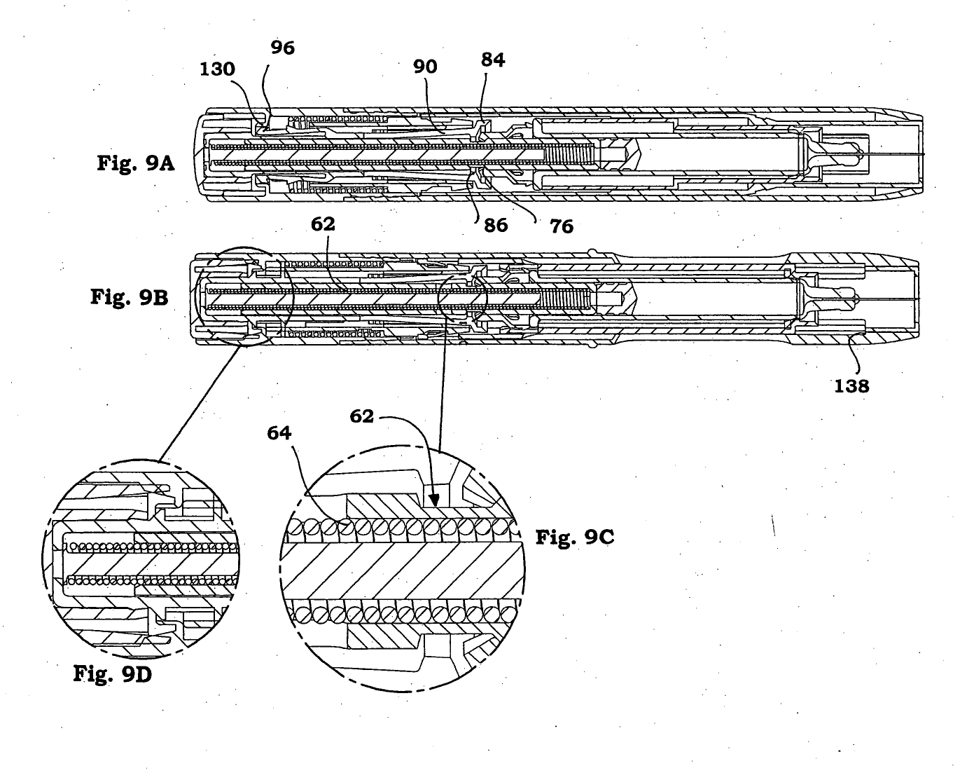

The next step is to activate the penetration and injection. Should the user however remove the injector from the injecting site the compression spring 122 will push the actuator sleeve 110 and thereby the needle shield 20 back to its original position and a press on the button will not cause the device to fire. When activating the penetration and injection, the user merely depresses the activator button, FIG. 9. This causes the activator to be moved to the right in the figures whereby the hooks 96 pass inside the circumferential ledge 130 and the band-shaped part 84 completely out of the actuator sleeve. The resilient properties of the tongues 90 of the activator causes the ledges 86 to move out of the groove 62 of the plunger, which then is free to move due to the spring 64. During the movement of the plunger, the ledges 76 of the holding member 68 are also moved out of the groove because the arms 74 of the holding member are no longer held in place by the band-shaped part of the activator.

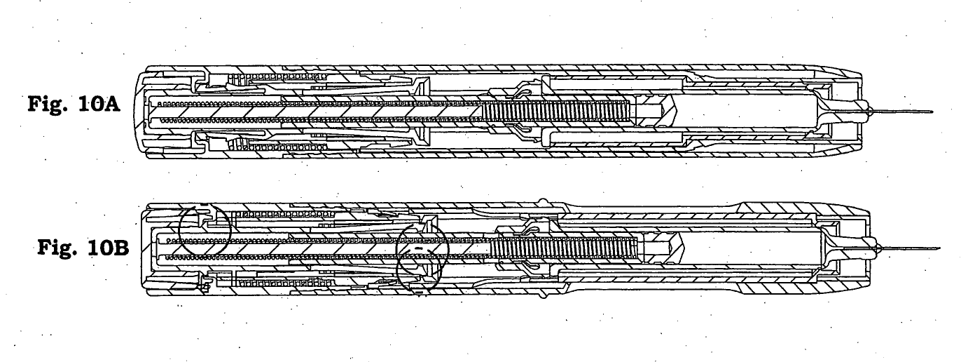

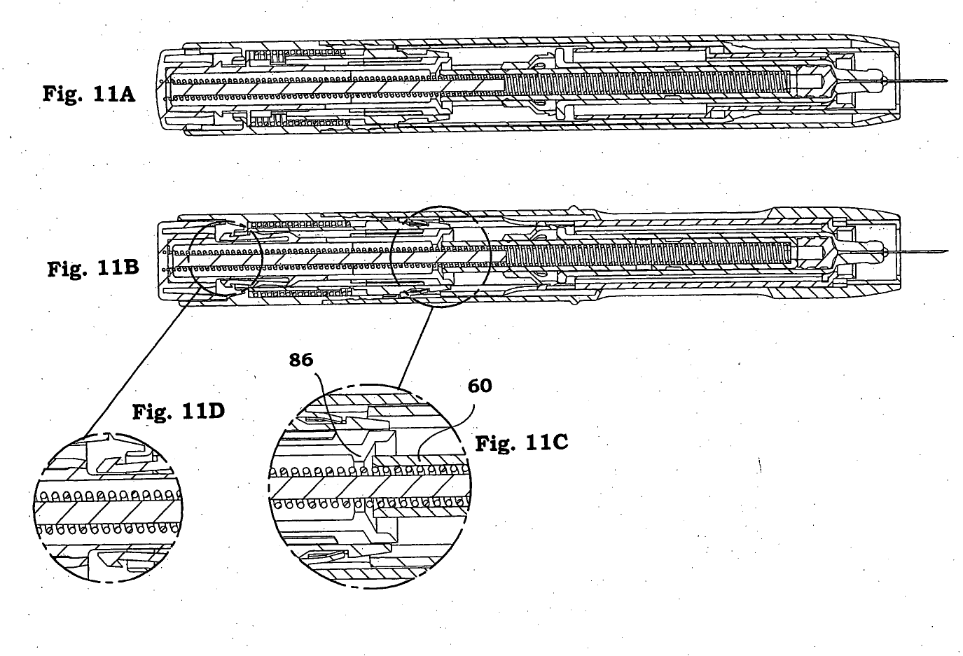

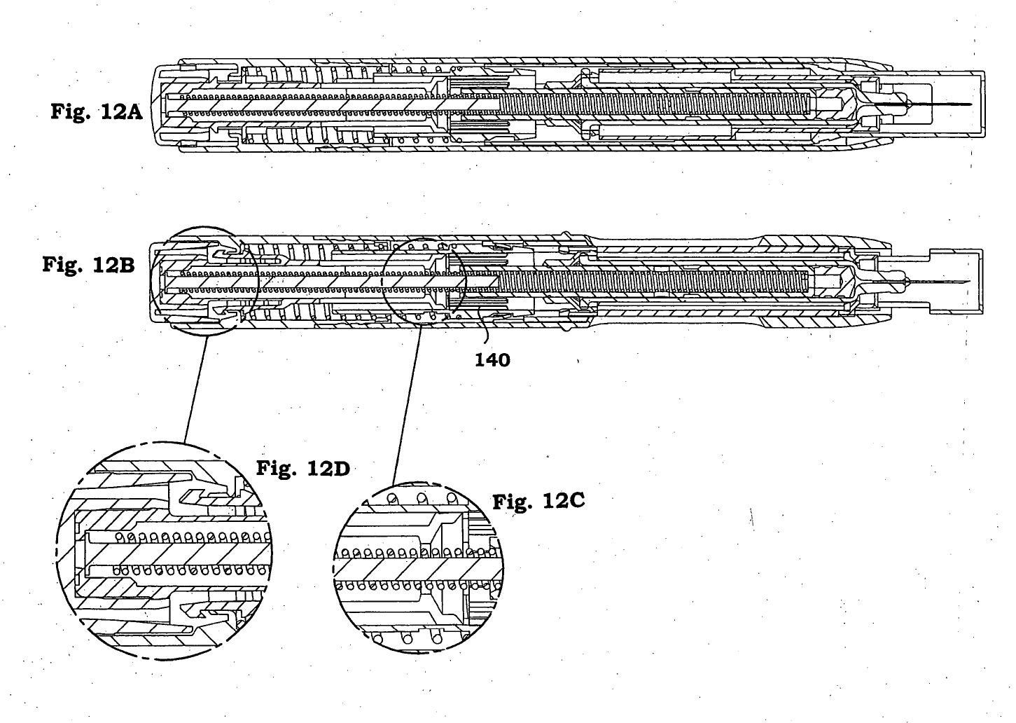

The force of the compression spring urges the plunger to push on the stopper of the syringe. But because of the friction between stopper and container wall and incompressibility of liquid in the syringe and the very small flow passage through the needle, the force will push the syringe forward, to the right in the figures, and thereby penetrate the skin of the patient, FIG. 10. The penetration stops when the front surface of the syringe carrier surrounding the neck portion abuts a ledge 138, FIG. 9B, arranged inside the front part. The force from the compression spring now moves the stopper inside the syringe and the liquid medicament is injected into the patient until the stopper reaches the inner front end of the syringe. When the plunger has moved this distance, its rear end has passed the ledges 86 of the activator and the tongues are moved inwards, FIG. 11C. Because the compression spring is also acting on the activator, the activator is moved inside the actuator sleeve. Because of this and because the needle shield spring is acting on the actuator sleeve it is urged forward, to the right in the figures. When now the injector is removed from the injection site, the force of the needle shield spring pushes the actuator sleeve and thus the needle shield connected to it forward, whereby the needle shield is pushed out of the front end of the injector and surrounds the needle. The movement of the actuator sleeve causes the band-shaped part 84 of the actuator 80 to pass ribs 140 arranged on the inner surface of the actuator sleeve, FIG. 12. These ribs prevent any attempts to push the needle shield back into the injector because the ribs will abut the front end of the band-shaped part 84 of the actuator 80. The needle shield is thus locked, which prevents unintentional needle sticks.

As a safety measure, it is not possible to first press the activator button 102 and then press the injector against an injection site and release a penetration/injection action because the depression of the activator button causes the hooks 96 to engage the circumferential ledge 130. Because of the inclined surfaces between the hooks 96 and the circumferential ledge 130 it is not possible to push the tongues 104 inwards by the actuator sleeve 110 acting on the inclined protrusions on the tongues. In order to release a penetration/injection action, the injector has to be pressed first against an injection site in order to be able to depress the activator button and release the plunger.

It is to be understood that the embodiment described above and shown in the drawings is to be regarded as a non-limiting example of the invention and that it is defined be the patent claims.

1. Injection device comprising a generally elongated tubular housing, a container containing medicament and having a needle, a needle shield slidably arranged to the housing and protruding a distance outside the front end of said housing, a plunger arranged to act on said container and a pre-tensioned drive means arranged to drive said plunger, characterized in that it comprises radially acting holding means capable of releasably holding said plunger, axially acting actuator means connected to said needle shield and capable of releasably locking said holding means and axially acting activator means capable of releasing said holding means from said actuator means when said needle shield and said actuator means has moved axially a certain distance due to that injection device has been placed against an injection site.

2. A device according to claim 1, characterized in that said holding means comprises generally radially extending inwards directed ledges and that said plunger comprises a groove, with mutual shapes as for the ledges to fit into said groove.

3. A device according to claim 2, characterized in that said ledges are arranged at the ends of movable tongues.

4. A device according to claim 2, characterized in that said actuator means comprises a sleeve having an inner diameter generally corresponding to the radial extension of the holding means in the area of the ledges.

5. A device according to claim 1, characterized in that it comprises a locking means capable of locking said activator means until said actuator means has moved axially a certain distance.

6. A device according to claim 5, characterized in that said locking means comprises hooks arranged on said activator means cooperating with fixedly arranged stop means and that said actuator means comprises means for displacing said hooks in relation to said stop means upon movement of said actuator means.

7. Device according to claim 1, characterized in that said holding means is arranged and designed such that said plunger is prevented from being released if said actuator means is operated before said needle shield is exposed to pressure.

8. Device according to claim 1, wherein said holding means further is arranged and designed such that said plunger is prevented from being released if said actuator means is operated at the same time as said needle shield is exposed to pressure.

9. A device according to claim 3, characterized in that said actuator means comprises a sleeve having an inner diameter generally corresponding to the radial extension of the holding means in the area of the ledges.

10. A device according to claim 2, characterized in that it comprises a locking means capable of locking said activator means until said actuator means has moved axially a certain distance.

11. A device according to claim 3, characterized in that it comprises a locking means capable of locking said activator means until said actuator means has moved axially a certain distance.

12. A device according to claim 4, characterized in that it comprises a locking means capable of locking said activator means until said actuator means has moved axially a certain distance.
